(19) United States
(12) Patent Application Publication
(10) Pub. No.: US 2019/0381170 A1
(43) Pub. Date: Dec. 19, 2019

(54) PROPHYLACTIC AND/OR THERAPEUTIC AGENT FOR AUTOIMMUNE DISEASE, AND VACCINE

(71) Applicant: INSTITUTE FOR RHEUMATIC DISEASES CO., LTD., Ashiya-shi (JP)

(72) Inventors: Kazuko SHIOZAWA, Ashiya-shi (JP); Shunichi SHIOZAWA, Ashiya-shi (JP)

(73) Assignee: INSTITUTE FOR RHEUMATIC DISEASES CO., LTD., Ashiya-shi (JP)

(21) Appl. No.: 16/489,108

(22) PCT Filed: Feb. 26, 2018
(86) PCT No.: PCT/JP2018/007012

§ 371 Date: Aug. 27, 2019

(30) Foreign Application Priority Data
Mar. 1, 2017 (JP) 2017-038810

(51) Int. Cl. A61K 39/395; A61P 37/02

(52) U.S. Cl. CPC A61K 39/395; A61P 37/02

(57) ABSTRACT

In order to provide a medical agent for use in prevention and/or treatment of an autoimmune disease, an agent is used which contains, as an active ingredient, an anti-DOCK8 antibody, a DOCK8 protein, or a partial sequence of the DOCK8 protein.

### TECHNICAL FIELD

The present invention relates to an agent for prevention and/or treatment of an autoimmune disease, and a vaccine for prevention and/or treatment of an autoimmune disease.

### BACKGROUND ART

An immune system originally exists as a defense mechanism of a biological body against invasion by harmful external foreign substances. However, in some cases, an action of the immune system turns out to be harmful to the biological body. It is known that a biological body has immune responses to not only external foreign substances but also self-components. Such an immune response to a self-component is called an autoimmune phenomenon. Further, a disease involving a specific pathological condition caused by autoimmunity is called an autoimmune disease.

Autoimmune diseases are systemic diseases, and classified broadly into two types: (i) organ-specific diseases (organ-specific autoimmune diseases) and (ii) non-organ-specific diseases (non-organ-specific autoimmune diseases). In many of these autoimmune diseases, tissue lesions are observed. At the same time, the autoimmune diseases are associated with tissue injuries.

It is known that systemic lupus erythematosus (SLE), which is a non-organ-specific autoimmune disease, is caused by an increased production of autoantibodies such as anti-dsDNA antibodies. There are expectations for implementation of molecular biological studies on a process of onset of SLE, for clarification of a relation between various pathological conditions and SLE, and further for development of treatment methods.

Repeated immunization with one kind of antigen on an experimental animal causes immunological response to reach an acme phase and eventually an exhaustion phase. It is known that as a result of this process, various autoimmune diseases (pathological conditions) associated with tissue injuries occur (see, for example, Patent Literature 1 and Non-Patent Literature 1).

Studies on tissue injuries caused by autoimmune diseases are currently being advanced. It has been clarified that cross-presentation of antigens by antigen-presenting cells is involved in tissue injuries. More specifically, the following are clarified: (1) a patient suffering from an autoimmune disease has an enhanced cross-presentation of antigens by dendritic cells; and (2) the enhanced cross-presentation by dendritic cells activates CD8⁺T cells and the CD8⁺T cells induce cytotoxicity (see for example, Patent Literature 2 and Non-Patent Literature 1).

The inventors of the present application have reported the following. As a result of repeated immunomanipulation with an antigen on mice, T cells of a new type, which are called autoreactive autoantibody-inducing CD4 T cells (aiCD4 T cells), are produced in the periphery. Then, the aiCD4 T cells induce various autoantibodies and also cause cytotoxic T cells (CTLs) to mature. As a result, various organ disorders occur and this consequently results in onset of SLE (Non-Patent Literature 1). Further, the inventors of the present application have found that (i) aiCD4 T cells are present in a CD4 T cell subpopulation of PD-1⁺ CD45RB^{low}122^{low} (PD-1⁺ CD45RB^{lo}122^{lo}), and (ii) two weeks after adoptive transfer of this cell subpopulation into naive mice, autoantibodies are observed in serum of those recipient mice (see Non-Patent Literature 2).

Furthermore, the inventors of the present application have found a marker for more simply identifying a cell which causes onset of an autoimmune disease, and have succeeded, on the basis of this finding, development of a technique which allows for an objective, precise diagnosis of the autoimmune disease (see Patent Literature 3).

### CITATION LIST

### Patent Literature

[Patent Literature 1]
Japanese Patent Application Publication Tokukai No. 2006-288382 (Publication date: Oct. 26, 2006)
[Patent Literature 2]
Japanese Patent Application Publication Tokukai No. 2010-004750 (Publication date: Jan. 14, 2010)
[Patent Literature 3]
Japanese Patent Application Publication Tokukai No. 2017-003329 (Publication date: Jan. 5, 2017)

### Non-Patent Literature

[Non-patent Literature 1]
Tsumiyama K. et al., PLoS ONE, Vol. 4, No. 12, e8382, 2009
[Non-patent Literature 2]
Miyazaki Y. et al., The Journal of Immunology vol. 192, no. 2, supplement 177.7, 2014
[Non-patent Literature 3]
Zhang Q et al., N Engl J Med 361(21): 2046-2055, 2009
[Non-patent Literature 4]
Randall K L et al., Nat Immunol 10(12): 1283-1291, 2009
[Non-patent Literature 5]
Randall K L et al. Disease Markers 29: 141-150, (2010)
[Non-patent Literature 6]
Randall K L et al., J Exp Med 208(11): 2305-2320, 2011
[Non-patent Literature 7]
Werner M and Jumaa H, Nat Immunol 13(6): 525-526, 2012
[Non-patent Literature 8]
Jabara H H et al., Nat Immunol 13(6): 612-620, 2012

### SUMMARY OF INVENTION

### Technical Problem

However, medical agents for use in prevention and/or treatment of autoimmune diseases are still insufficient and further development of novel medical agents has been desired.

An object of an embodiment of the present invention is to provide a novel medical agent for use in prevention and/or treatment of an autoimmune disease.

### Solution to Problem

As a result of diligent studies, the inventors of the present application have found that symptoms of an autoimmune disease can be improved with the use of an anti-DOCK8 antibody, and have accomplished the present invention. The knowledge obtained by the inventors of the present application suggests involvement of a DOCK8 protein in a process of onset of the autoimmune disease. The knowledge was surprising in the field to which the present application pertains.

In order to solve the above problem, an agent for prevention, treatment, or both prevention and treatment of an autoimmune disease in accordance with an aspect of the present invention, contains an anti-DOCK8 antibody as an active ingredient.

In order to solve the above problem, a vaccine for prevention, treatment, or both prevention and treatment of an autoimmune disease in accordance with an aspect of the present invention, contains, as an active ingredient, a DOCK8 protein, a partial sequence of the DOCK8 protein, an expression vector of the DOCK8 protein, or an expression vector of the partial sequence of the DOCK8 protein.

### Advantageous Effects of Invention

An aspect of the present invention makes it possible to prevent and/or treat an autoimmune disease.

### BRIEF DESCRIPTION OF DRAWINGS

FIG. 1 is a graph showing test results of proteinuria in Examples of the present invention.

FIG. 2 is an image of test results with regard to localization of DOCK8 protein in Examples of the present invention.

### DESCRIPTION OF EMBODIMENTS

The following description will discuss an embodiment of the present invention, but the present invention is not limited to the following embodiments. Note that the present invention is not limited to any of configurations described below, but can be altered by a skilled person in the art within the scope of the claims. The present invention also encompasses, in its technical scope, any embodiment or example derived by combining technical means disclosed in differing embodiments and/or examples. All academic and patent literatures listed herein are incorporated herein by reference. Any numerical range expressed as “A to B” herein means “not less than A and not more than B” unless otherwise stated.

1. Agent for Prevention and/or Treatment of Autoimmune Disease

As described above, the inventors of the present application have previously reported a technique for diagnosing an autoimmune disease by checking, as an indicator, an increase of CD4 T cells (DOCK8-positive CD4 T cells (hereinafter, also referred to as DOCK8⁺ CD4 T cells)) which express dedicators of cytokinesis protein 8 (hereinafter, referred to as DOCK8) (Patent Literature 3). However, it has not been clear by what mechanism the DOCK8⁺ CD4 T cells are involved in a process of onset of the autoimmune disease. In light of the above, the inventors of the present application have made diligent studies and found that, surprisingly, an antibody against DOCK8 has an autoimmune disease preventing/treating effect. It should be however noted that even if it has been found that the presence of the DOCK8⁺ CD4 T cells could serve as an indicator for diagnosis of an autoimmune disease, there is still a very large gap between finding this fact and developing a treatment agent.

An agent for prevention and/or treatment of an autoimmune disease in accordance with an embodiment of the present invention, contains an anti-DOCK8 antibody as an active ingredient.

The term “autoimmune disease” herein refers to a disease causing a pathological condition associated with autoimmunity. From a patient suffering an “autoimmune disease”, an autoantibody (e.g., anti-Sm antibody, anti-dsDNA antibody, and rheumatoid factor (RF)) against a self-component (e.g., immunoglobulin) is detected. Examples of the autoimmune disease encompass: organ-specific autoimmune diseases (such as chronic thyroiditis, primary myxedema thyrotoxicosis, pernicious anemia, Goodpasture's syndrome, rapidly progressive glomerulonephritis, myasthenia gravis, pemphigus vulgaris, bullous pemphigoid, insulin-resistant diabetes, juvenile diabetes, Addison's disease, atrophic gastritis, male infertility, climacterium precox, lens-induced uveitis, sympathetic arteritis, multiple sclerosis, ulcerative colitis, primary biliary cirrhosis, chronic active hepatitis, autoimmune hemolytic anemia, paroxysmal hemoglobinuria, idiopathic thrombocytopenic purpura, and Sjogren's syndrome) and non-organ-specific autoimmune diseases (such as rheumatoid arthritis, systemic lupus erythematosus (SLE), discoid lupus erythematosus, polymyositis, scleroderma, and mixed connective tissue disease). Among the above, rheumatoid arthritis, systemic lupus erythematosus, discoid lupus erythematosus, polymyositis, scleroderma, and mixed connective tissue disease are known as collagen disease. In other words, collagen disease is also included in systemic autoimmune diseases. The autoimmune disease in accordance with one aspect of the present invention is preferably collagen disease, and more preferably, systemic lupus erythematosus.

DOCK8 protein is a member of DOCK family proteins, and serves as a guanine nucleotide exchange factor (GEF). The DOCK8 protein has a molecular weight of 238 kDa. Further, the DOCK8 protein is known to control polymerization of actin cytoskeleton through activation of a Rho family low-molecular-weight G protein. Remodeling of actin cytoskeleton is involved in various cell functions such as cell differentiation, cell multiplication, cell migration and cell signaling.

In recent years, it has been discovered that a defect in human DOCK8 gene causes severe combined immunodeficiency, and this has led to reports of immunological study results on DOCK8 (see Non-Patent Literatures 3 to 8).

Note that “DOCK8” herein refers to both the DOCK8 protein and a DNA (gene) encoding the DOCK8 protein, unless otherwise stated.

An anti-DOCK8 antibody binds to a DOCK8 protein or a partial sequence of the DOCK8 protein. In this case, the origin of the DOCK8 protein to which the anti-DOCK8 antibody binds is not particularly limited. The DOCK8 protein can originate from, for example, mammals. Examples of the mammals encompass mice, rats, rabbits, goats, monkeys and humans. The mammals are preferably mice, rats, or humans, and more preferably humans.

In Examples described later, it is suggested that the DOCK8 protein present at a surface of a cell membrane is involved in a mechanism of onset of SLE. Therefore, the anti-DOCK8 antibody in accordance with an embodiment of the present invention can be an antibody which inhibits localization of the DOCK8 protein on a cell membrane. For example, suppose that the DOCK8 protein is localized on cell membranes of cells A, and an arbitrarily selected antibody is added to some cells A, which are referred to as cells B. Then, both the cells A and the cells B are immunostained with the anti-DOCK8 antibody. In a case where staining intensity in the vicinity of cell membranes of the cells B is lower than that of the cells A, it can be determined that the arbitrarily selected antibody is an antibody which inhibits localization of the DOCK8 protein on cell membranes.

More specifically, the anti-DOCK8 antibody can be an antibody which binds to a human-derived polypeptide consisting of an amino acid sequence of SEQ ID NO: 1 or 5, a mouse-derived polypeptide consisting of an amino acid sequence of SEQ ID NO: 2, or a partial sequence of each of these polypeptides.

The number of amino acids constituting the partial sequence is not particularly limited, and can be, for example, not more than 400, not more than 350, not more than 300, not more than 200, not more than 100, not more than 90, not more than 80, not more than 70, not more than 60, not more than 50, not more than 40, not more than 30, not more than 20, or not more than 10. Further, the lower limit of the number of amino acids constituting the partial sequence is not particularly limited, and can be, for example, 3, 4, 5, 6, 7, 8, 9, or 10.

The anti-DOCK8 antibody is an antibody which specifically binds to a DOCK8 protein or a partial sequence of the DOCK8 protein. Note that the expression that an antibody “specifically binds to” a DOCK8 protein or a partial sequence of the DOCK8 protein means that the antibody binds with a stronger affinity to a DOCK8 protein or a partial sequence of the DOCK8 protein than to other polypeptides. The wording “with a stronger affinity” means, for example, an affinity having a binding constant (Ka) of not less than 10⁷ M⁻¹, preferably not less than 10⁸ M⁻¹, more preferably not less than 10⁹ M⁻¹, more preferably not less than 10¹⁰ M⁻¹, more preferably not less than 10¹¹ M⁻¹, more preferably not less than 10¹² M⁻¹, and most preferably not less than 10¹³ M⁻¹.

The anti-DOCK8 antibody can be an antibody containing a whole anti-DOCK8 antibody, an antibody consisting of the whole anti-DOCK8 antibody, an antibody containing a fragment of the anti-DOCK8 antibody, or an antibody consisting of a fragment of the anti-DOCK8 antibody. For example, the DOCK8 antibody can be a polyclonal antibody, a monoclonal antibody, a chimeric antibody, a humanized antibody, a human antibody, or a fragment of any of these antibodies (e.g., F(ab′)₂, Fab′, Fab, or Fv). For example, in a case where the anti-DOCK8 antibody is a monoclonal antibody, it is possible to reduce non-specific binding of the anti-DOCK8 antibody to a target other than the DOCK8 protein. This makes it possible to enhance a pharmacological effect of the agent for prevention and/or treatment of an autoimmune disease in accordance with an embodiment of the present invention, and also makes it possible to prevent the occurrence of side effects of the agent for prevention and/or treatment of an autoimmune disease in accordance with an embodiment of the present invention. The anti-DOCK8 antibody can be IgA, IgD, IgE, IgG, IgM, or a fragment of any of these antibodies.

The anti-DOCK8 antibody can be prepared by a well-known method (e.g., (1) HarLow et al., “Antibodies: A laboratory manual, Cold Spring Harbor Laboratory, New York (1988)” or (2) Iwasaki et al., “Monoclonal antibody hybridoma and ELISA, KODANSHA (1991)”). It is certainly possible to use a commercially-available antibody as the anti-DOCK8 antibody.

The monoclonal antibody can be prepared by a well-known technique (e.g., (1) a hybridoma technique (Kohler, G. and Milstein, C., Nature 256, 495-497 (1975)), (2) a trioma technique, (3) a human B-cell hybridoma technique (Kozbor, Immunology Today 4, 72 (1983)) or (4) an EBV-hybridoma technique (Monoclonal Antibodies and Cancer Therapy, Alan R Liss, Inc., 77-96 (1985))) in the field to which the present application pertains.

The monoclonal antibody can be prepared also by a phage display technique. In a case where the monoclonal antibody is prepared by the phage display technique, it is possible to use a publicly known method. The following will discuss one example of a method for preparing the monoclonal antibody by the phage display technique. First, mRNA is prepared from an animal immunized with the DOCK8 protein. Then, cDNA is prepared by using the mRNA as a template, and further, a single-stranded antibody (scFV) gene encoding only a variable region of that antibody is prepared from the cDNA. Next, the single-stranded antibody gene is inserted into a phagemid vector. Subsequently, after this phagemid vector is introduced into E. coli, the E. coli is infected with a phage. This allows scFV antibodies to be expressed on a phage membrane. Among the scFV antibodies which have been expressed on the phage membrane, antibodies which bind to the DOCK8 protein are screened. As a result, the anti-DOCK8 monoclonal antibody can be prepared. Note that well-known methods can be used for mRNA preparation, cDNA preparation, insertion of the single-stranded antibody gene into the phagemid vector, introduction of the phagemid vector into E. coli, infection with the phage, and antibody screening.

The chimeric antibody refers to an antibody having a constant region replaced by a constant region of a human antibody. The chimeric antibody can be prepared by a well-known method (see, for example, European Patent Application Publication No. EP0125023).

The humanized antibody refers to an antibody in which regions except for complementarity determining regions (CDRs) in an H chain and an L chain are replaced by a human antibody structure.

The human antibody refers to an antibody prepared by using a transgenic animal into which a gene involved in human antibody production is introduced (see, for example, European Patent Application Publication No. EP0546073).

It is possible to obtain F(ab′)₂, Fab′, Fab, and Fv by (i) degrading a whole antibody with the use of protease (e.g., papain or pepsin) and (ii) further reducing a product of that degradation as needed. Alternatively, it is possible to obtain F(ab′)₂, Fab′, Fab, and Fv by (i) isolating cDNA of F(ab′)₂, Fab′, Fab, and Fv from a hybridoma which produces an antibody, (ii) inserting the cDNA into an expression vector, and (iii) introducing the expression vector into a host. In this case, it is possible to obtain an antibody fragment as a fusion protein of the antibody fragment and another protein.

The anti-DOCK8 antibody contained in the agent for prevention and/or treatment of an autoimmune disease in accordance with an embodiment of the present invention is not particularly limited in amount relative to an amount of the agent for prevention and/or treatment. For example, the amount of the anti-DOCK8 antibody with respect to 100% by weight of the agent for prevention and/or treatment can be 0.001% by weight to 100% by weight, 0.01% by weight to 100% by weight, 0.1% by weight to 100% by weight, 0.1% by weight to 95% by weight, 0.1% by weight to 90% by weight, 0.1% by weight to 80% by weight, 0.1% by weight to 70% by weight, 0.1% by weight to 60% by weight, 0.1% by weight to 50% by weight, 0.1% by weight to 40% by weight, 0.1% by weight to 30% by weight, 0.1% by weight to 20% by weight, or 0.1% by weight to 10% by weight.

The anti-DOCK8 antibody contained in the agent for prevention and/or treatment of an autoimmune disease in accordance with an embodiment of the present invention is not particularly limited in amount. For example, the amount of the anti-DOCK8 antibody can be 0.01 mg to 1000 mg, 0.1 mg to 1000 mg, 1 mg to 1000 mg, 1 mg to 900 mg, 1 mg to 800 mg, 1 mg to 700 mg, 1 mg to 600 mg, 1 mg to 500 mg, 1 mg to 400 mg, 1 mg to 300 mg, 1 mg to 200 mg, or 1 mg to 100 mg.

Further, the agent for prevention and/or treatment of an autoimmune disease in accordance with an embodiment of the present invention can contain a component(s) (e.g., a pharmaceutically acceptable carrier) other than the anti-DOCK8 antibody. For example, in a case where the agent for prevention and/or treatment of an autoimmune disease in accordance with an embodiment of the present invention is a solid preparation, examples of the component(s) other than the anti-DOCK8 antibody encompass an excipient, a lubricant, a binder, and a disintegrant. On the other hand, in a case where the agent for prevention and/or treatment of an autoimmune disease in accordance with an embodiment of the present invention is a liquid preparation, examples of the component(s) other than the anti-DOCK8 antibody encompass a solvent, a solubilizing agent, a suspension, a tonicity agent, a buffer, and a soothing agent. In addition, examples of the component(s) other than the anti-DOCK8 antibody also encompass an antiseptic, an antioxidant, and a stabilizer.

Examples of the “excipient” encompass lactose, saccharose, D-mannitol, xylitol, sorbitol, erythritol, starch, and crystalline cellulose. However, the “excipient” is not limited to these.

Examples of the “lubricant” encompass magnesium stearate, calcium stearate, wax, talc, and colloid silica. However, the “lubricant” is not limited to these.

Examples of the “binder” encompass α-starch, methyl cellulose, crystalline cellulose, saccharose, D-mannitol, trehalose, dextrin, hydroxypropyl cellulose, hydroxypropyl methylcellulose, and polyvinyl pyrrolidone. However, the “binder” is not limited to these.

Examples of the “disintegrant” encompass starch, carboxymethyl cellulose, low substituted hydroxypropylcellulose, carboxymethylcellulose calcium, croscarmellose sodium, and carboxymethyl starch sodium. However, the “disintegrant” is not limited to these.

Examples of the “solvent” encompass water for injection, alcohol, propylene glycol, macrogol, sesame oil, corn oil, and tricaprilin. However, the “solvent” is not limited to these.

Examples of the “solubilizing agent” encompass polyethylene glycol, propylene glycol, D-mannitol, trehalose, benzyl benzoate, ethanol, trisaminomethane, cholesterol, triethanolamine, sodium carbonate, and sodium citrate. However, the “solubilizing agent” is not limited to these.

Examples of the “suspension” encompass surface-active agents (e.g., stearyl triethanolamine, sodium lauryl sulfate, lauraminopropionic acid, lecithin, benzalkonium chloride, benzethonium chloride, and glyceryl monostearate) and hydrophilic polymers (e.g., polyvinyl alcohol, polyvinyl pyrrolidone, carboxymethylcellulose sodium, methyl cellulose, hydroxymethyl cellulose, hydroxyethyl cellulose, and hydroxypropyl cellulose). However, the “suspension” is not limited these.

Examples of the “tonicity agent” encompass sodium chloride, glycerin, and D-mannitol. However, the “tonicity agent” is not limited to these.

Examples of the “buffer” encompass phosphate, acetate, carbonate, and citrate. However, the “buffer” is not limited to these.

Examples of the “soothing agent” encompass benzyl alcohol. However, the “soothing agent” is not limited to this.

Examples of the “antiseptic” encompass p-hydroxybenzoate esters, chlorobutanol, benzyl alcohol, phenethyl alcohol, dehydroacetic acid, and sorbic acid. However, the “antiseptic” is not limited to these.

Examples of the “antioxidant” encompass sulfite and ascorbic acid. However, the “antioxidant” is not limited to these.

Further, the “stabilizer” is not particularly limited, and can be any stabilizer that is normally employed in the field of pharmaceuticals.

The component(s) other than the anti-DOCK8 antibody, which component(s) is/are contained in the agent for prevention and/or treatment of an autoimmune disease in accordance with an embodiment of the present invention, is/are not particularly limited in amount relative to an amount of the agent for prevention and/or treatment. For example, the amount of the component(s) other than the anti-DOCK8 antibody with respect to 100% by weight of the agent for prevention and/or treatment can be 0% by weight to 99.999% by weight, 0% by weight to 99.99% by weight, 0% by weight to 99.9% by weight, 5% by weight to 99.9% by weight, 10% by weight to 99.9% by weight, 20% by weight to 99.9% by weight, 30% by weight to 99.9% by weight, 40% by weight to 99.9% by weight, 50% by weight to 99.9% by weight, 60% by weight to 99.9% by weight, 70% by weight to 99.9% by weight, 80% by weight to 99.9% by weight, or 90% by weight to 99.9% by weight.

The component(s) other than the anti-DOCK8 antibody contained in the agent for prevention and/or treatment of an autoimmune disease in accordance with an embodiment of the present invention is/are not particularly limited in amount. For example, the amount of the component(s) other than the anti-DOCK8 antibody can be 0.01 mg to 1000 mg, 0.1 mg to 1000 mg, 1 mg to 1000 mg, 1 mg to 900 mg, 1 mg to 800 mg, 1 mg to 700 mg, 1 mg to 600 mg, 1 mg to 500 mg, 1 mg to 400 mg, 1 mg to 300 mg, 1 mg to 200 mg, or 1 mg to 100 mg.

The agent for prevention and/or treatment of an autoimmune disease in accordance with an embodiment of the present invention can be administered by any method. The agent for prevention and/or treatment of an autoimmune disease can be administered orally or alternatively, administered parenterally (intravenously, rectally, intraperitoneally, intramuscularly, intranasally, or subcutaneously).

The agent for prevention and/or treatment of an autoimmune disease in accordance with an embodiment of the present invention is not particularly limited in dosage form. For example, the dosage form of the agent for prevention and/or treatment of an autoimmune disease can be an injection (e.g., intraperitoneal injection, intravenous injection, subcutaneous injection, intracutaneous injection, intramuscular injection, or infusion), a collunarium, or a suppository.

2. Vaccine for Prevention and/or Treatment of Autoimmune Disease

As described in Examples later, it is possible to prevent and/or treat an autoimmune disease with the use of the anti-DOCK8 antibody. This indicates that it is also possible to prevent and/or treat an autoimmune disease with the use of a vaccine containing a substance (e.g., antigen) which can induce production of the anti-DOCK8 antibody when administered into a biological body.

Therefore, a vaccine for prevention and/or treatment of an autoimmune disease in accordance with an embodiment of the present invention, contains, as an active ingredient, a DOCK8 protein, a partial sequence of the DOCK8 protein, an expression vector of the DOCK8 protein, or an expression vector of the partial sequence of the DOCK8 protein.

For example, the vaccine for prevention and/or treatment of an autoimmune disease in accordance with an embodiment of the present invention contains, as an active ingredient, a DOCK8 protein derived from a species which is different from that of an administration target, a partial sequence of such a DOCK8 protein, an expression vector of the DOCK8 protein derived from a species that is different from that of an administration target or an expression vector of the partial sequence of such a DOCK8 protein.

The vaccine for prevention and/or treatment of an autoimmune disease in accordance with an embodiment of the present invention and the above-described agent for prevention and/or treatment of an autoimmune disease are different from each other only in substance used as the active ingredient and are identical to each other in constituents except for the substance used as the active ingredient. The vaccine for prevention and/or treatment of an autoimmune disease in accordance with an embodiment of the present invention has been described above except for that active ingredient, and therefore, an explanation thereof is omitted here.

The DOCK8 protein and the partial sequence of the DOCK8 protein as active ingredients each can be a human-derived polypeptide consisting of an amino acid sequence of SEQ ID NO: 1 or 5, a mouse-derived polypeptide consisting of an amino acid sequence of SEQ ID NO: 2, or a partial sequence of each of these polypeptides.

The number of amino acids constituting the partial sequence is not particularly limited, and can be, for example, not more than 400, not more than 350, not more than 300, not more than 200, not more than 100, not more than 90, not more than 80, not more than 70, not more than 60, not more than 50, not more than 40, not more than 30, not more than 20, or not more than 10. Further, the above partial sequence only needs to be a partial sequence of the DOCK8 protein, and can be at any position in the DOCK8 protein.

The DOCK8 protein and the partial sequence of the DOCK8 protein each can be prepared by a publicly known method. For example, the DOCK8 protein and the partial sequence of the DOCK8 protein each can be prepared by: (i) using a publicly-known peptide synthesizer; or (ii) introducing, into a desired host (e.g., E. coli or yeast), an expression vector into which cDNA encoding the DOCK8 protein or cDNA encoding the partial sequence of the DOCK8 protein has been inserted.

The expression vector of the DOCK8 protein or the expression vector of the partial sequence of the DOCK8 protein, as an active ingredient, is an expression vector in which cDNA encoding the DOCK8 protein or cDNA encoding the partial sequence of the DOCK8 protein is inserted in an expressible manner.

Specific examples of the vector are not particularly limited, and encompass plasmids, virus vectors (e.g., vaccinia virus vectors, herpesvirus vectors, adenovirus vectors, adeno-associated virus vectors, and retrovirus vectors), or virus particles. Specific examples of a promoter to be inserted into the vector are not particularly limited, and can be selected as appropriate depending on the vector as described above.

In the vector, cDNA encoding the DOCK8 protein or cDNA encoding the partial sequence of the DOCK8 protein is inserted in an expressible manner. The cDNA is not particularly limited in specific base sequence. Examples of the cDNA encoding the DOCK8 protein or the cDNA encoding the partial sequence of the DOCK8 protein encompass a human-derived polynucleotide consisting of a base sequence of SEQ ID NO: 3, a mouse-derived polypeptide consisting of a base sequence of SEQ ID NO: 4, and a partial sequence of each of these polypeptides.

The active ingredient contained in the vaccine for prevention and/or treatment of an autoimmune disease in accordance with an embodiment of the present invention is not particularly limited in amount relative to an amount of the vaccine for prevention and/or treatment. For example, the amount of the active ingredient with respect to 100% by weight of the vaccine for prevention and/or treatment can be 0.001% by weight to 100% by weight, 0.01% by weight to 100% by weight, 0.1% by weight to 100% by weight, 0.1% by weight to 95% by weight, 0.1% by weight to 90% by weight, 0.1% by weight to 80% by weight, 0.1% by weight to 70% by weight, 0.1% by weight to 60% by weight, 0.1% by weight to 50% by weight, 0.1% by weight to 40% by weight, 0.1% by weight to 30% by weight, 0.1% by weight to 20% by weight, or 0.1% by weight to 10% by weight.

The active ingredient contained in the vaccine for prevention and/or treatment of an autoimmune disease in accordance with an embodiment of the present invention is not particularly limited in amount. For example, the amount of the active ingredient can be 0.01 mg to 1000 mg, 0.1 mg to 1000 mg, 1 mg to 1000 mg, 1 mg to 900 mg, 1 mg to 800 mg, 1 mg to 700 mg, 1 mg to 600 mg, 1 mg to 500 mg, 1 mg to 400 mg, 1 mg to 300 mg, 1 mg to 200 mg, or 1 mg to 100 mg.

Each aspect of the present invention can be configured as below.

An agent for prevention, treatment, or both prevention and treatment of an autoimmune disease in accordance with an aspect of the present invention, contains an anti-DOCK8 antibody as an active ingredient.

The agent for prevention, treatment, or both prevention and treatment of an autoimmune disease in accordance with an aspect of the present invention is preferably configured such that: the autoimmune disease is systemic lupus erythematosus (SLE).

The agent for prevention, treatment, or both prevention and treatment of an autoimmune disease in accordance with an aspect of the present invention is preferably configured such that: the anti-DOCK8 antibody binds to a polypeptide consisting of an amino acid sequence of SEQ ID NO: 1, 2, or 5 or a partial sequence of the polypeptide.

The agent for prevention, treatment, or both prevention and treatment of an autoimmune disease in accordance with an aspect of the present invention is preferably configured such that: the anti-DOCK8 antibody is a monoclonal antibody.

A vaccine for prevention, treatment, or both prevention and treatment of an autoimmune disease in accordance with an aspect of the present invention, contains, as an active ingredient, a DOCK8 protein, a partial sequence of the DOCK8 protein, an expression vector of the DOCK8 protein, or an expression vector of the partial sequence of the DOCK8 protein.

### Examples

[Test 1]

To BALB/c mice (Charles River Japan Inc.), 0.5 mg of egg albumin (OVA: grade V; Sigma) or PBS was intraperitoneally administered every 5 days repeatedly. This prepared mice exhibiting a symptom of an autoimmune disease.

Further, to the BALB/c mice, 50 μg of a rabbit-derived anti-DOCK8 antibody or 50 μg of a control IgG (rabbit IgG) was intraperitoneally administered 24 hours before 6th, 9th, and 12th administration of OVA and 6th, 9th, and 12th administration of PBS.

Note that the anti-DOCK8 antibody used in Test 1 was a commercially available anti-DOCK8 polyclonal antibody (proteintech, DOCK8 Polyclonal Antibody, Catalog No. 11622-1-AP, Antibody: polypeptide consisting of an amino acid sequence of SEQ ID NO: 5).

After the 12th administration of OVA and after the 12th administration of PBS, proteinuria was detected by a color reaction with the use of ALBSTIX (registered trademark) (SIEMENS). The proteinuria is an indicator of onset of nephritis which is caused by systemic lupus erythematosus. Further, Hematoxylin-Eosin (HE) staining was performed by a well-known method. Then, pathological evaluation of kidneys was performed according to a WHO classification. Specifically, the pathological evaluation of kidneys was performed by classifying observed kidney symptoms into classes I-II, class III, class IV, and class V according to the criteria described in “Weening J J et al., The classification of glomerulonephritis in systemic lupus erythematosus revised. J Am Soc Nephrol 15(2):241-250, 2004”.

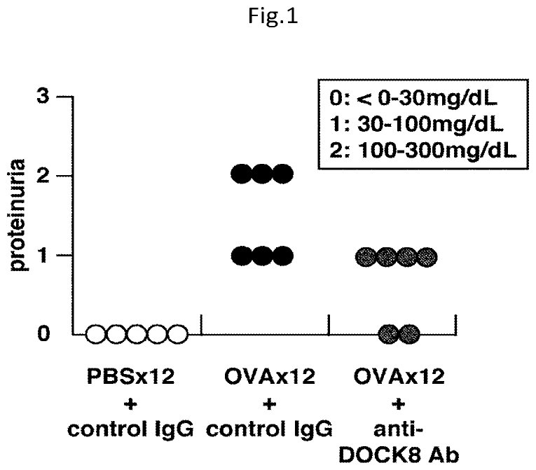

FIG. 1 shows test results of proteinuria. Table 1 below shows pathological evaluation of kidneys according to the WHO classification.

As shown in FIG. 1, proteinuria was detected in a BALB/c mouse group (“OVA×12+control IgG” in FIG. 1) to which the control IgG had been administered before the 6th, 9th, and 12th administration of OVA. In contrast, in another BALB/c mouse group (“OVA×12+anti-DOCK8 Ab” in FIG. 1) to which the anti-DOCK8 antibody had been administered before the 6th, 9th, and 12th administration of OVA, a decrease in level of proteinuria was observed. Note that “PBS×12+control IgG” in FIG. 1 shows a test result of an yet another BALB/c mouse group to which the control IgG had been administered before the 6th, 9th, and 12th administration of PBS.

Further, as shown in Table 1, in the BALB/c mouse group (“OVA×12+control IgG” in Table 1) to which the control IgG had been administered before the 6th, 9th, and 12th administration of OVA, severe nephritis such as nephritis of WHO classes IV and V was detected. In contrast, in the another BALB/c mouse group (“OVA×12+anti-DOCK8 Ab” in Table 1) to which the anti-DOCK8 antibody was administered before the 6th, 9th, and 12th administration of OVA, nephritis was substantially completely prevented. Note that “PBS×12+control IgG” in Table 1 shows a test result of the yet another BALB/c mouse group to which the control IgG had been administered before the 6th, 9th, and 12th administration of PBS.

TABLE 1

| | WHO class | | | |
|---|---|---|---|---|
| | < II | III | IV | V |
| PBSx12 + control IgG | 55.71 ± 6.06% | 22.87 ± 4.04% | 13.57 ± 5.05% | 7.85 ± 3.03% |
| OVAx12 + control IgG | 5.94 ± 5.87% | 17.93 ± 8.04% | 22.41 ± 5.80% | 53.71 ± 11.24% |
| OVAx12 + | 48.34 ± 21.02% | 32.63 ± 10.71% | 13.46 ± 6.63% | 5.54 ± 7.99% |
| anti-DOCK8 Ab | | | | |

[Test 2]

To BALB/c mice (Charles River Japan Inc.), 0.5 mg of egg albumin (OVA: grade V; Sigma) was intraperitoneally administered every 5 days repeatedly. This prepared mice exhibiting a symptom of an autoimmune disease.

Further, to the BALB/c mice, 50 μg of a rabbit-derived anti-DOCK8 antibody (which is the same as the rabbit-derived anti-DOCK8 antibody used in [Test 1]) or 50 μg of control IgG (rabbit IgG) was intraperitoneally administered 24 hours before 6th, 9th, and 12th administration of OVA.

After the 12th administration of OVA, autoantibodies in serum (specifically, rheumatoid factor (RF), anti-Sm Ab, and anti-dsDNA Ab) were detected with the use of an enzyme-linked immunosorbent assay (ELISA).

Table 2 below shows test results of the ELISA.

As shown in Table 2, as compared to a BALB/c mouse group (“OVA×12+control IgG” in Table 2) to which the control IgG had been administered before the 6th, 9th, and 12th administration of OVA, all three kinds of autoantibodies significantly decreased in another BALB/c mouse group (“OVA×12+anti-DOCK8 Ab” in FIG. 3) to which the anti-DOCK8 antibody was administered before the 6th, 9th, and 12th administration of OVA.

TABLE 2

| | OVAx12 + | OVAx12 + | |
|---|---|---|---|
| | control IgG | anti-DOCK8 Ab | |
| | (n = 5) | (n = 5) | |
| RF (U/ml) | 74.14 ± 14.61 | 45.14 ± 23.49 | P < 0.05 |
| anti-Sm Ab (AU) | 0.53 ± 0.07 | 0.40 ± 0.10 | P < 0.05 |
| anti-dsDNA Ab (AU) | 0.63 ± 0.04 | 0.57 ± 0.02 | P < 0.05 |

[Test 3]

To BALB/c mice (Charles River Japan Inc.), 0.5 mg of egg albumin (OVA: grade V; Sigma) was intraperitoneally administered every 5 days repeatedly. This prepared mice exhibiting a symptom of an autoimmune disease.

After 12th administration of OVA, T cells were fractionated into some subpopulations. Then, a cytoplasmic fraction and a membrane fraction were obtained by a well-known method from T cells in each subpopulation.

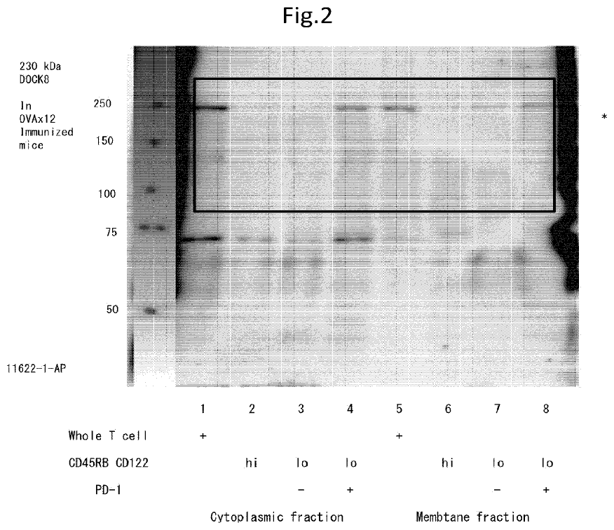

Thereafter, 10 μg of the cytoplasmic fraction per lane and 10 μg of the membrane fraction per lane were subjected to electrophoresis (10% polyacrylamide gel), so that proteins contained in each of these fractions were isolated. Subsequently, a DOCK8 protein contained in the proteins thus isolated was detected by Western blotting with the use of an anti-DOCK8 antibody. Results of this detection are shown in FIG. 2.

In FIG. 2, lanes 1 and 5 show test results of Whole T cell-derived cytoplasmic fraction and membrane fraction, respectively. Moreover, lanes 2 and 6 show test results of CD45RB^{high}122^{high} cell-derived cytoplasmic fraction and membrane fraction, respectively. Further, lanes 3 and 7 show test results of PD-1⁻ CD45RB^{low}122^{low} cell-derived cytoplasmic fraction and membrane fraction, respectively. In addition, lanes 4 and 8 show test results of PD-1⁺ CD45RB^{low}122^{low} cell-derived cytoplasmic fraction and membrane fraction, respectively.

Among the above cells, PD-1⁺ CD45RB^{low}122^{low} cells contain autoreactive autoantibody-inducing CD4 T cells (aiCD4 T cells). Further, in FIG. 2, an image which is observed at around 230 kDa corresponds to the DOCK8 protein.

As is clear from the lanes 4 and 8 in FIG. 2, in the PD-1⁺ CD45RB^{low}122^{low} cells, not only the cytoplasmic fraction but also the membrane fraction contained a lot of DOCK8 proteins. This indicates that in the D-1⁺ CD45RB^{low}122^{low} cells, a lot of DOCK8 proteins are localized at surfaces of cell membranes. Note that also based on the fact that the PD-1⁺ CD45RB^{low}122^{low} cells can be recognized by a flow cytometry with the use of the anti-DOCK8 antibody, it can be considered that in D-1⁺ CD45RB^{low}122^{low} cells, a lot of DOCK8 proteins are localized at surfaces of cell membranes.

It has been known, from past research, that because aiCD4 T cells contained in a CD4 T cell subpopulation (specifically, PD-1⁺ CD45RB^{low}122^{low}) induce various autoantibodies and also cause cytotoxic T cells (CTLs) to mature, various organ disorders occur and this consequently results in onset of SLE. Therefore, the test results of the present application suggest that not only the DOCK8 protein present in the cytoplasmic fraction but also the DOCK8 protein present at the surface of the cell membrane are involved in a mechanism of onset of SLE.

### INDUSTRIAL APPLICABILITY

An aspect of the present invention is applicable to prevention and/or treatment of an autoimmune disease.

1. A method of preventing, treating, or both preventing and treating an autoimmune disease, comprising:
administering an agent for prevention, treatment, or both prevention and treatment of an autoimmune disease, the agent comprising an anti-DOCK8 antibody as an active ingredient.

2. The method of claim 1, wherein:
the autoimmune disease is systemic lupus erythematosus (SLE).

3. The method of claim 1, wherein:
the anti-DOCK8 antibody binds to a polypeptide consisting of an amino acid sequence of SEQ ID NO: 1, 2, or 5 or a partial sequence of the polypeptide.

4. The method of claim 1, wherein:
the anti-DOCK8 antibody is a monoclonal antibody.

5. A method of preventing, treating, or both preventing and treating an autoimmune disease, comprising:
administering a vaccine for prevention, treatment, or both prevention and treatment of an autoimmune disease, the vaccine comprising a DOCK8 protein, a partial sequence of the DOCK8 protein, an expression vector of the DOCK8 protein, or an expression vector of the partial sequence of the DOCK8 protein, as an active ingredient.


SEQUENCE LISTING (5 sequences)

SEQ ID NO: 1 - protein, 2099 aa, Homo sapiens [sequence omitted]

SEQ ID NO: 2 - protein, 2100 aa, House mouse
MATLPSAERRAFALKINRYSSSEIRKQFTLPPNLGQYHRHSISTSGFPSLQLPQLYEPVEPVDFEGLVMTHLNSLDAELAQELGDLTDDDLHVAFTPKECRTLQHSLPEEGVELDPHVRDCVQTYIREWLIVNRKNQGSSEFCSFKKTGSRRDFQKTLQKQTFESETLECSEPDTQTGPRHPLNVLCDVSGKGPLTSCDFDLRSLQPDERLENLLQLVSAEDFEKEKEEARKTNRPAELFALYPPVDEEDAVEIRPVPECPKEHLGNRILVKVLTLKFEIEIEPLFASIALYDVKERKKISENFHCDLNSDQFKGFLRAHTPSIDPSSQARSAVFSVTYPSSDIYLVVKIEKVLQQGEIADCAEPYMIIKESDGGKSKEKVEKLKLQAESFCQRLGKYRMPFAWAPISLASFFNISTLERESTDVEPGVGRNSVGEKRSLSQSRRPSERTLSLEENGVGSNFKATTLATNIFFKQEGDRLSDEDLFKFLADYKRSSSLQRRVKSIPGSLRLEISPAPDVMNCCLTPEMLPVKPFPENRTRPHKEILEFPIREVYVPHTVYRNLLYVYPQRLNFASKLASARNITIKIQFMCGEDPSNAMPVIFGKSSGPEFLQEVYTAITYHNKSPDFYEEVKIKLPAKLTVNHHLLFTFYHISCQQKQGASGESLLGYSWLPILLNERLQTGSYCLPVALEKLPPNYSIHSAEKVPLQNPPIKWAEGHKGVFNIEVQAVSSVHTQDNHLEKFFTLCHSLESQVSFPIRVLDQKITESTLEHELKLSIICLNSSRLEPLVLFLHLVLDKLFQLSVQPMVIAGQTANFSQFAFESVVAIANSLHNSKDLRKDQHGRNCLLASYVHYVFRLPELHRDTSKSGGPITVVPDPRYHTYGRTSAAAVSSKLMQARVMSSSNPDLTGSHCAADEEVKNIMSSKIADRNCSRMSYYCSGNSDAPGSTAAPRPVSKKHFHEELALQMVVSTGVVRETVFKYAWFFFELLVKSMAQYVHNLDKRDSFRRTRFSDRFKDDITTIVNVVTSEIAALLVKPQKESEQAEKINISLAFFLYDLLSIMDRGFVFNLIKHYCSQLSAKLNILPTLISMRLEFLRILCSHEHYLNLNLLFMNTDTAPASPCPSISSQNSSSCSSFQDQKIASMFDLTPEYRQQHFLTGLLFTELAVALDAEGDGISRVQRKAVSAIHSLLCSHDLDPRCRKPEVKVKIAALYLPLVGIILDALPQLYDFTDARSGRSRASGSYEEQDVANGINQNVALAIAGNHFNLKTSGAMLSSLPYKQYNMLNADTTRHLMICFLWIMKNADQSLIRKWIADLPSMQLNRILDLLFICVSCFEYKGKQSSDKVSNQVLQKSRDVKAKLEEALLRGEGARGEMMRRRIPGTDRFPGINENLRWRKEQTQWRQANEKLDKTKAELDQEALISGNLATEANLIILDMQENIIQASSALDCKDSLLGGVLRVLVNSLSCDQSTTYLTHCFATLRALIAKFGDLLFEEEMEQCADLCQRVLHHCSSSMDVTRSQACATLYLLMRFSFGATSNFARVKMQVTMALASLVGKAPDFNEEHLRRSLRTILAYSEEDTAMQTTPFPMQVEELLCNLNSILYDTVKMREFQEDPEMLMDLMYRIAKSYQASPDLRLTWLQNMAEKHTKKKCFTEAAMCLVHAAALVAEYLSMLEDHSYLPVGSVSFQNISSNVLEESAVSDDTLSPDEDGVCSGRYFTESGLVGLLEQAAELFSTGGLYETVNEVYKLVIPILEAHRDFRKLTSTHDKLQKAFDNIINKDHKRMFGTYFRVGFYGSRFGDLDEQEFVYKEPAITKLPEISHRLEGFYGQCFGAEFVEVIKDSTPVDKTKLDPNKAYIQITFVEPYFDEYEMKDRVTYFEKNFNLRRFMYTTPFTLEGRPRGELHEQHRRNTVLTTMHAFPYIKTRIRVSQKEEFVLTPIEVAIEDMKKKTLQLAVATHQEPPDAKMLQMVLQGSVGATVNQGPLEVAQVFLAEIPADPKLYRHHNKLRLCFKEFIMRCGEAVEKNRRLITAEQREYQQELKKNYNKLRDSLRPMIERKIPELYKPIFRVDSQKRDSFHRSSFRKCETQLSQGS

SEQ ID NO: 3 - DNA, 7448 bp, Homo sapiens
gacagacgaggtttgcgcttggctgggcatgttccgcggctactctgcggcgcgccaggcccccgctttccgcaccccgcgaccctagaagccaccgaaccgccggcgggccatggccactctgccgagcgcagagcgccgcgcgttcgcgctcaagatcaacaggtattcttcagtggaaataaggaaacagtttactctcccaccaaaccttggccagtaccatcgacagagcataagtacctctggcttcccctctcttcaactacctcagttttatgaccctgtggagccagtggactttgaaggacttctgatgacacacctgaacagcctggatgtgcagcttgcccaggagctcggggacttcactgatgacgacttggacgtggtgttcacgacaaaggaatgtaggactttgcagccctctttgccggaggaaggggttgaactggaccctcatgtcagggactgtgttcagacctacatccgtgagtggctaatcgtgaaccggaaaaaccaaggaagtccagaaatctgtggctttaaaaagactggatctcgaaaagattttcacaagacgcttccgaaacagacgtttgagtcggaaaccttggagtgcagtgaacccgctgctcaggcaggcccccgccacttaaacgtgctgtgcgacgtgtctgggaaaggccccgtcactgcctgtgactttgacctccgcagcctgcagcctgacaagcggctagaaaacctcctgcagcaagtgagtgccgaggactttgagaagcagaacgaggaggcccggaggaccaataggcaggccgagctctttgccctttacccatcagtggacgaggaggatgctgtggaaatacgtccagtaccagaatgtcccaaggaacacctgggcaacagaatattggtcaagttgctgaccttgaagttcgagattgaaattgagcccctgtttgccagcattgccctctacgatgttaaagaaaggaaaaagatctcagaaaattttcactgtgacctgaactctgaccagttcaaaggatttctgcgagctcacacgccttcagtggccgcatcaagtcaggcgagatctgcagtcttctcagtcacctacccgtcctcagacatctacctggtagtcaagattgaaaaagtcctgcagcagggagagattggagactgtgcagagccctacacggttatcaaagaaagtgatggtggaaagagtaaagaaaagattgaaaaactaaaactccaagctgaatccttctgccagcgtttggggaaataccggatgccctttgcctgggcacccataagcttatcaagcttcttcaatgtctccacccttgagagggaggtaactgatgtggactctgtggttgggagaagctcagtgggtgaacggaggacattggcccaatctagaaggctttctgaaagagccctctccttggaggaaaatggggttggatccaacttcaaaacctccactctgagcgttagcagctttttcaagcaggaaggagatcgccttagcgatgaagacttattcaagtttttagctgactacaaaagatcatcatccttacagagacgagtcaagtcaattccaggcttgctaagactggagatttctacagctccagagatcatcaattgctgtctgactcctgaaatgctgcccgtgaaaccctttcctgaaaaccggacacgcccgcacaaagagattttggaatttccaacacgagaagtatatgtccctcacactgtgtacagaaaccttctctatgtctacccacagaggctgaactttgtaaacaaactagcatcagcccggaacattacaataaagatccagtttatgtgtggagaagatgctagcaatgcgatgccggtcatctttggaaagtccagcgggcctgaatttctgcaggaagtgtacacagctgttacataccataataagtctcctgacttttatgaagaagtgaaaattaagctccccgctaagctcacagtaaatcaccacctcctgttcaccttctaccatatcagctgtcagcagaagcaaggagcctccgtggaaactctcctgggatattcatggctgccaattctcttaaatgaacgtcttcaaactggatcctactgtctcccagttgccttggaaaaattgccacccaactactccatgcattctgctgagaaagtcccattacagaatcctcccattaagtgggctgaaggacataagggagtatttaatattgaagtgcaagctgtttcttctgtacacacccaggacaaccacctggagaagttcttcaccctctgccactccctggagagccaggtgaccttccccatccgcgtgctggatcagaaaatcagcgagatggcgctggagcatgagctgaagctcagcatcatctgcctcaactcctcccgcctggagccgctcgtgctcttcctgcacctggtgctggacaagctcttccagctgtccgtgcagcccatggtcatcgctggccagacagccaacttctcccagtttgccttcgagtccgtggtggccatcgccaacagtctgcacaacagcaaggacctgagcaaggaccagcatgggaggaactgcctgctggcttcctacgtgcactacgtcttccgcctgccagaggtgcaaagggatgtgcccaagtcaggcgctcccactgccctcctagaccctcggagctaccacacgtatggccgcacatcagctgctgctgtgagttcaaagctgctgcaggcccgggtgatgagcagcagtaacccagacctcgcggggacacactccgcagcagacgaggaagtgaagaacatcatgtcttcaaagatcgccgatcgcaactgcagccgaatgtcttactattgctctggcagtagtgatgctccaagttcacctgcagccccaaggccagccagcaaaaagcatttccatgaggagcttgcccttcagatggtggtcagcaccggaatggtgagagaaacagtcttcaagtatgcctggttcttctttgagcttctggtgaaaagcatggcccagcacgtacataacatggacaaacgggacagttttcggaggactcgtttttctgaccgtttcatggatgacataactactattgttaatgtggtcacctcggaaattgcagcccttttagtaaaaccacagaaggaaaatgaacaggcggaaaagatgaacatcagcctggctttcttcttgtatgaccttctctccctcatggatcggggctttgtgtttaacctcatcagacattattgcagccagctgtcagccaagctcagtaaccttccaacgctcatttccatgaggctagagttcctgagaatcctctgtagccatgagcattacctcaatctgaaccttttttttatgaatgctgatactgctccaacatctccttgtccttccatatcttcccagaactcaagctcctgctccagcttccaggaccagaagatcgccagcatgttcgatctgacttccgagtaccgccagcagcacttcctcaccgggctcctcttcacagaactggctgctgccctggatgccgaaggggaaggaatcagcaaagtacaaaggaaagctgtcagtgcaattcacagcctgctaagttctcacgacctggacccacgctgtgtcaaaccagaggtgaaggtcaaaatcgccgccctttacctacctttagttggcatcattttggatgctttgccacagctctgtgactttacagttgcagatactcgcagataccgcaccagtggctcggatgaagaacaagaaggagccggtgccattaaccagaatgtggctctggccatagcagggaataatttcaatttgaaaacaagtggaatagtgctgtcttccttgccctataagcagtacaacatgctgaacgcggacactactcgcaacctcatgatctgcttcctctggatcatgaaaaatgctgatcagagcctcattaggaagtggattgctgacctgccatcaacgcagctcaacaggattttagatctacttttcatctgtgtgttatgttttgagtataagggaaaacagagttctgacaaagtcagtacccaagtcctgcagaagtcaagggatgtcaaggcccggctggaagaggctttgctccgtggggaaggggccagaggggagatgatgcgccgccgggctccagggaacgaccgatttccaggcctaaatgaaaatttgagatggaagaaagagcagacacattggcggcaagctaatgagaagctagataaaacaaaggccgagttagatcaagaagccttgatcagtggcaatctggctacagaagcacatttaatcatcctggatatgcaggaaaacattatccaggcgagctcggctctggactgtaaagacagcctgctgggaggtgttctgagggtgctggtgaattctctgaactgtgatcagagtaccacctacctgactcactgctttgcaacactccgtgctctcatcgccaagtttggagacttactcttcgaagaggaggtggaacagtgtttcgacctatgtcaccaagtcctgcaccactgcagcagcagcatggatgtcacccggagccaagcctgtgccaccctttacctcctcatgaggttcagttttggagccaccagtaattttgcaagagtaaagatgcaagtaaccatgtccctggcatctttggtgggaagagcaccagactttaatgaagagcacctgagaagatccttgaggacaattttggcctattcagaagaggacacagccatgcagatgactccttttcccacccaggtggaggaacttctctgtaatctgaatagcatcttatatgacacagtgaaaatgagggaatttcaggaagatcctgagatgcttatggatctcatgtacagaattgccaagagttaccaggcatctcctgatctgcggctgacctggctccagaacatggcagagaaacacaccaagaagaagtgctacacggaggctgccatgtgcctggtgcacgccgctgcgttagtggctgagtatctgagcatgctggaggaccacagctacctgcccgtgggcagtgtcagcttccagaatatttcttccaatgtgctggaggagtctgtggtctctgaggacaccctgtcacctgacgaggatggggtgtgcgcaggccagtacttcaccgagagtggcctggtaggcctcctggagcaggccgcggagctcttcagcacgggaggcttatatgagacagttaatgaggtctacaagctggtcatccccatcctagaagcgcatcgagaattccggaagctgacactcactcacagcaagctgcagagagccttcgacagcatcgttaacaaggatcataagagaatgtttggaacctacttccgagttggtttctttggatccaaatttggggatttggatgaacaggagtttgtctacaaagagcctgcaattaccaagcttcctgagatctcacatagactagaggcattttatggtcaatgttttggtgcagaatttgtggaagtgattaaagactccactcctgtggacaaaaccaagttggatcctaacaaggcctacatacagatcacttttgtggagccctactttgatgagtatgagatgaaagacagggtcacatactttgagaagaatttcaacctccggaggttcatgtacaccaccccgttcaccctggaggggcggcctcggggagagctgcatgagcagtacagaaggaacacagtcctgaccactatgcacgccttcccctacatcaagaccaggatcagcgtcatccagaaggaggagtttgttttgacaccgattgaagttgccattgaagacatgaagaagaagaccctgcagttagcagttgccattaaccaggagccgcctgatgcaaagatgcttcagatggtgctgcaaggctctgtgggagctactgtaaatcagggaccactggaagtagcccaagtgtttttggctgaaattcctgctgatccaaaactctatcgacatcacaacaagttgaggttatgctttaaggaattcatcatgagatgtggtgaagctgtagagaaaaacaagcgtctcatcacggcagaccagagggaatatcagcaggaactcaaaaagaactataacaagctaaaagagaacctcaggccaatgatcgagcggaaaattccagaactgtacaagccaatattcagagttgagagtcaaaagagggactccttccacagatctagtttcaggaaatgtgaaacccagttgtcacagggcagctaagaaaagccatcttcattcgtggagactgtggccctgcaaccctggagaaggacttgctggtacttaaaaaatgggacatttgccacccaggactgactgtacactccctgatcagccagcactctggaagctttgggatcccaggaaccatggaattattcccaaatggactctgaccagatttttgccatactggggggtggcgggatggaggatgggtactcaggcatgactgcgtatttattaaagtgtgtttttccacaatgtaccaaacaaggcataagcagcttctcctgctgactggccaatcactgcccatctgagagatgatttcctctggcccatatttgaatttattggagtaactcaaattgcctgaggaaaaatggaaaaattatccaccagtcgattcaaactgaatttcactctttataggaaggcagggcaaacttgtaggagtacgaaacattttcaataaatctacaaagggaagccttactacaattccaaaaatcatcatggttggaaatttgggaggagattatttgtgaacttgttacccttttggtaatggtggactaattgctgtatagttatttttgttttattattactgttacattaatttaacatgcatttatagaagaatacattcaaagcactgatgtaggagatacacggtacttggagcagtcagccagaaatcacagatactgctttcacttaaatggaaacaattctccgataatgctttgctttttttcttatgtcactcttgtgtactatctatttttctcctctctgggaccaagtttctttttataaagcaataatatctctgttttcatttcagaacattgtgctgtctgtcagcatatgtatatcagctacaaaatatattcaactttgacttcttttgacaaaggactttaggaaaaagaggaacaaagacattatttgagaattaaattatatatttttaatatgactgtgaccttgactgataataaagatgtaataagaattgcaagctaaa

SEQ ID NO: 4 - DNA, 7651 bp, House mouse
gcggctgacagatgaggctggcgccgggcgcgccgggctctgcagcggctgtgcgttcgggtcccgtccccccgcagccccgcgagccccgaagccgtggaaccgcgcgcgcgcgccatggccacgctgccgagcgccgagcgccgcgccttcgcgctcaagatcaacaggtattcatcgtcagaaataaggaagcagtttacgctcccacccaacctcggacagtaccatcggcacagtatcagtacatctggtttcccctctcttcagctacctcagctttatgagcctgtcgagccagtggactttgaaggactcgtgatgacacacttaaacagcttggatgcagagctggcccaggagctgggggacctcaccgatgacgacctgcatgtggccttcacacccaaagaatgtaggactttgcagcactctctgccagaggaaggagttgaactggatcctcacgtcagagactgtgttcagacctatattcgagagtggctgattgtaaaccggaaaaaccaaggaagttcagagttttgcagctttaaaaagacgggatctcgcagagattttcagaagacgcttcagaaacagacgtttgagtcagaaaccttggagtgcagtgaaccggacactcagacaggaccccgtcatcccttaaacgtgctgtgtgacgtgtctgggaagggccccctcacttcttgtgacttcgacctccgcagcctgcagcctgatgagcggctggaaaacctgctccagcttgtgagcgctgaggactttgagaaggagaaggaggaggcccgcaagaccaatcggccggctgagctctttgccctctatccgcccgtggatgaggaggatgctgtggaaatacgtccagtacctgaatgtcctaaggaacatctgggcaacagaatattggtcaaggtgctgactctgaagtttgagattgaaattgaacctctgtttgccagtattgccctctatgacgttaaagaaaggaaaaagatctcagaaaatttccactgtgacctgaactccgaccagttcaaagggtttctgcgagctcacacaccctcgattgacccatcgagtcaggctaggtctgccgtgttctctgtcacctacccatcttcagacatctacctggttgtcaagattgaaaaggtccttcagcaaggagagattgcagactgtgcagaaccctacatgatcatcaaagaaagcgatggtggaaagagtaaagaaaaggttgaaaaactaaaacttcaagctgaatccttctgccaacgtttggggaaataccggatgcccttcgcctgggcccccattagcttagcaagcttcttcaacatctccacccttgaaagggagagcacagatgtggagcctggggttgggaggaactctgtgggtgagaagaggagcttgtcccaatccaggaggccctctgagcgaaccctctccttggaggaaaatggagttggatccaacttcaaagccaccaccttggccaccaacatcttcttcaaacaggagggagatcgccttagtgatgaagacttgttcaagtttttagctgactacaagagatcttcatccctacagcgaagagtcaaatccatcccaggctcactgaggctggagatatccccagctcccgacgtgatgaactgctgcctgacgcccgagatgctgccagtcaaaccttttcctgaaaatcggacgcgtccacacaaggagattttggaatttccgatccgggaggtgtacgtccctcacactgtgtacagaaaccttctgtacgtatacccccagcgactgaacttcgctagcaagctagcatctgcccggaacatcacaataaagattcagtttatgtgcggagaagaccccagcaatgctatgccggtcatctttggcaagtccagtgggcctgaatttctgcaggaagtatatacagctattacataccataataagtctcctgacttttacgaagaagtgaaaattaagctccctgccaagctcacagtgaatcatcacctcctcttcaccttctaccacatcagctgtcagcagaagcaaggggcctccggagaaagccttctggggtactcgtggctgccgattctgttaaacgaacgtcttcaaaccggatcctactgtctgcctgttgccttggaaaaactaccacccaactactccatacattctgctgagaaagtccctttacagaatcctcccattaagtgggccgagggccataagggagtatttaatattgaagtgcaagctgtttcttccgtccacacccaggataaccacctggagaagttcttcaccctttgccactccctggagagccaggtgagcttccctatccgtgtgctggaccagaagatcaccgagagcacgctggagcacgagctgaaactcagcatcatctgcctcaactcctcccgcctggagcccctcgtgctcttcctccacctggtgctcgacaagctgttccagctttccgtgcagcccatggtcattgctggccaaacagcaaacttctcccagtttgccttcgagtctgtggtggccattgccaatagcctgcacaacagcaaggacctgaggaaggaccagcacggaaggaactgccttctggcctcctatgtgcactacgtgttccggctgccggaactgcacagggatacatccaagtcaggtggccccatcaccgtagtccccgacccccgataccacacatatggacgcacatctgccgctgcagtgagttcaaagctgatgcaggcccgtgtgatgagcagcagcaacccagacctgactgggtcacactgtgcagccgatgaggaagttaagaacatcatgtcttcaaagattgccgatcgcaactgcagccggatgtcttactattgctctggcaatagtgatgcgccaggttcaactgcagccccaagaccagtcagcaaaaagcatttccatgaggagcttgccctgcagatggtggtcagcactggagtagtgagagaaacggtcttcaagtacgcctggttcttctttgagcttctggtgaaaagcatggcgcagtatgtccataacctggataaacgggacagttttcggaggactcgtttttctgaccgcttcaaagatgacataactaccattgttaatgtggtcacctcggagatagcagcccttttagtgaaacctcagaaggaaagcgagcaggcagaaaagatcaacatcagccttgccttcttcctgtatgacctcctgtcaatcatggacagaggcttcgtgttcaacctcatcaagcattactgcagccagctgtcagccaagctgaatatccttccaacgctcatctccatgcggctggaattcctgaggatcctctgcagccatgagcactacctcaacttgaacctcctcttcatgaataccgacaccgcaccagcatctccctgcccctccatatcctcccagaactcgagttcctgctccagtttccaggaccaaaagattgccagcatgttcgatctgaccccggagtaccggcagcagcacttccttacagggctgctcttcacggagctggctgttgccctggatgctgagggggatggaattagcagagtacagagaaaagccgtgagtgccatccacagccttctgtgttctcacgacctggatccacggtgtcgcaaaccggaagtgaaagtcaaaatcgccgccctttacctgccgttggtcggcatcattctggacgctctgccacagctctatgactttacagatgctcgcagtggaaggagtcgtgccagtggctcgtatgaagaacaagatgtggccaacggaatcaaccagaatgtggccctggccatagcggggaatcactttaatttgaagaccagtggagcaatgctgtcttccttgccctataagcagtacaacatgctgaatgcagacaccacccgccacctcatgatttgcttcctgtggatcatgaaaaatgctgatcagagcctcatcaggaagtggatcgctgacctgccttccatgcagctcaataggattctggacctgctgttcatctgtgtctcctgctttgaatacaagggaaagcagagttctgacaaagtcagtaaccaggtcctgcagaagtcaagagacgtcaaggccaagctggaagaggccctgctccgtggggaaggagcccgtggggagatgatgcgccgtcgcattccagggactgaccggtttccaggcataaatgaaaatctgagatggaggaaagagcagacacagtggcggcaggctaatgagaagctggacaaaacaaaggcagagttagatcaagaagccttgatcagtggcaacctggctacagaagctaatttgatcatcctggatatgcaggagaacatcatccaggcaagctccgccctggactgtaaagacagcctgctgggaggtgtcctccgggtcctggtgaattctctgagctgtgaccagagcaccacctacctgactcactgtttcgcaaccctccgagccctcatcgccaagtttggagacctgctgttcgaggaggagatggagcagtgtgctgacctgtgtcagcgggtgctacatcactgcagcagcagcatggacgtcacacggagccaagcctgcgccaccctctacctcctcatgcggttcagcttcggagccaccagtaactttgcaagggtaaagatgcaagtgaccatggcactggcatccctggtaggcaaagcaccagacttcaacgaagagcacctgagaaggtccttaaggacaattttggcctattcagaagaggacacggccatgcagacaactccttttcccatgcaggtggaggaacttctctgcaatctgaacagcattctgtacgacacagtgaagatgagggaattccaggaagaccctgagatgcttatggacctcatgtacagaattgccaagagctaccaggcatcgcctgacctgcggctgacttggctccagaacatggcagagaaacacactaagaagaagtgcttcacagaggccgccatgtgcctggtgcatgcagccgccctggtggccgagtacctgagcatgctggaggaccacagctacctgccggtgggcagcgtcagctttcagaatatttcttccaatgtgcttgaggagtctgcagtctctgatgacaccttgtcacctgatgaggacggcgtatgctctggtcggtacttcactgagagtggcctggtgggcctcctggagcaggctgcggagctcttcagcacgggaggcttgtacgagacggttaatgaagtctacaagctggtcatccctatcctggaggcacacagagatttccggaagctgacctccactcacgacaagctgcagaaggccttcgataacatcatcaacaaggaccataagaggatgtttgggacctacttccgagttggtttctacggatcccgatttggggatttggatgagcaggagttcgtgtacaaggaacccgcaatcacgaagctcccggagatctcacatagactagagggattttatggccagtgtttcggtgcagagtttgtggaagtgataaaagactctactccagtggacaaaaccaagttggatcctaacaaggcctacattcagatcacttttgtggagccttactttgatgaatatgagatgaaagaccgggtgacctacttcgagaagaatttcaacctccggaggttcatgtacaccacccccttcaccctggaggggagaccccggggcgagcttcatgagcaacaccgcagaaacaccgtgctcaccaccatgcacgccttcccctacatcaagaccaggatccgagtcagccagaaagaggagttcgttttgactccgattgaggttgccattgaagatatgaagaagaagaccctgcagttagccgtggccactcaccaggagccccctgatgcaaagatgctgcaaatggtactgcagggctctgtaggagccactgtaaatcagggaccactggaggtggcccaagtgttcttggctgaaattccagctgacccaaagctctaccgacatcacaacaagctgaggttgtgcttcaaggagttcataatgcgatgcggagaggccgtggagaagaacaggcgactcatcaccgcagagcagcgggagtaccagcaggagctgaagaagaactacaacaagctgagagacagcctcaggcccatgattgagcggaaaatcccagagctctacaagcccatattcagagttgacagtcagaagagggactctttccacagatctagtttcaggaaatgtgaaacccagttgtcacagggcagctgaaaaaagccacctttgccggtgacgactggggccccactacctggaaggactcgctggtacgtccacagcagccacccaagactgactccacagtccctgcccagccagtgctctggaaacttggggggtctatgaccacagaatgatacccaagtgtattgtggactatcttgaccatgtcaggggttggggtggggtgggtaaatcaggggataattgtatttattaaagtatttttcacagtgctgtagaaggaaggcctaagctgtttctgctgagtggcttggctggtcacaggcctcccagaaagtgacttcttctgacctatgtttgaattcctaagataaactcaaattgtggacagaagagtggaacactgtccagcaacctattccgagtgaatttcactcatccctttggtgagaagaaaagataaaactccagaagagtgaaccattttccaatatatctgtgagaaaaagtcttacaggaaatagatggagcgctccacacctctcctgagctgccggactatgtgccgtctagttattgctgcattgttgcttctgttaagctgagtgagtccacctctacgcaggggacagttgtacgagacagacagtccaggtcgcccggccacacagcactgacagcacatgccccagagcgggagcagttctcctaggacatcttgccggttccctgtacacttgctctcactgcctcctttttctccgggataaggtcatttttctacaagacattcatttcagaacattgtgtcctctgtgtgctacagggatatatgctttggattgtttttacagtgaacttggaagagggaaagaaacctatggtttgagaattaaattatatattttcatacaactcatgaccttacctaatgctaaagacagaataagaatgataagcaagtggggtgacaatgcacgcctttagtcccagcactcagaagacagagacaggtggatctctgagtttaaggccagcctggtctacagagcgagttccaggacagccagattacacaatgagaccatgtctcataaataaataaataaataaataaacaaacaaataaataaataaataaataaattgcaaaatgcttccctgtgccactgctgtttgtgactttgacaactcaaagtgctgtgaagcttcagagaccctggtaggactggccttagtcaagcagtgccctccagag

SEQ ID NO: 5 - protein, 350 aa, Artificial Sequence
  Artificially Synthesized Sequence
EVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS